(19) United States
(12) Patent Application Publication
(10) Pub. No.: US 2016/0194260 A1
(43) Pub. Date: Jul. 7, 2016

(54) METHOD FOR PRODUCTION OF P-CYMENE

(71) Applicant: Holmen AB, Stockholm (SE)

(72) Inventors: Jörg Brucher, Domsjö (SE); David Blomberg Saitton, Domsjo (SE)

(73) Assignee: Holmen AB, Stockholm (SE)

(21) Appl. No.: 14/912,046

(22) PCT Filed: Aug. 12, 2014
(86) PCT No.: PCT/SE2014/050934

§ 371 Date: Feb. 12, 2016

(30) Foreign Application Priority Data
Aug. 14, 2013 (SE) 1350954-2

(51) Int. Cl. C07C 5/367; C07C 5/22; C07C 15/02

(52) U.S. Cl. CPC C07C 5/367; C07C 15/02; C07C 5/22; C07C 2527/128

(57) ABSTRACT

The present invention relates to a method for production of p-cymene from cyclic monoterpenes in the presence of an Fe(III)-salt as a catalyst. In particular it relates to a method for production of p-cymene from cyclic monoterpenes containing high levels of sulphur, such as crude sulphate turpentine. The method can be performed as two different, subsequent processes for the isomerization and oxidation reactions, or in a single process wherein these reactions take place at the same time.

The present invention relates to a method for production of p-cymene from cyclic monoterpenes in the presence of an Fe(III)-salt as a catalyst. In particular it relates to a method for production of p-cymene from cyclic monoterpenes containing high levels of sulphur, such as crude sulphate turpentine. The method can be performed as two different, subsequent processes for the isomerization and oxidation reactions, or in a single process wherein these reactions take place at the same time.

### BACKGROUND

Cymene is a naturally occurring aromatic organic compound which structure consists of a benzene ring substituted with a methyl group and an isopropyl group. The structure of cymene is similar to the numerous monoterpenes containing a cyclohexene or cyclohexadiene ring but in contrast to those and other monoterpenes, cymene is a stable compound not undergoing the typical reactions of terpenes. The most common geometric isomer is p-cymene, in which the alkyl groups are para-substituted. There also exist two less common geometric isomers: o-cymene, in which the alkyl groups are ortho-substituted, and m-cymene, in which they are meta-substituted. p-Cymene and m-cymene are valuable base chemicals which for example are used in fragrances, pharmaceuticals, herbicides, dyes, and heat transfer media. Another industrially important use of p-cymene is as a starting material for p-cresol production via the Hock-Lange synthesis pathway. p-Cymene has additionally been proposed as a suitable ingredient in aviation fuel formulations. Compared to other aromatics used in automotive fuel formulations, such as benzene, toluene or ethyl benzene, p-cymene has lower toxicity and is degraded easier in both aquatic and terrestrial systems.

Turpentine from boreal hard- and softwood species is a complex mixture of different terpenes, with the monoterpenes α-pinene, β-pinene and 3-carene as main constituents. As sterical strained, unsaturated hydrocarbons, terpenes are highly reactive compounds that easily undergo rearrangements, di- or trimerisation reactions or oxidation reactions. During the sulphate pulping process, terpenes stay unaltered and are condensed together with methanol from the off-gases. The turpentine is separated from other liquids by decantation, forming the typical crude sulphate turpentine (CST). Dominating impurities in CST are methanol along with organic sulphur compounds, polysulphides, and elementary sulphur. Turpentine is almost insoluble in water and thus CST and other turpentines generally contain only small amounts of water, such as less than 1%.

Monoterpenes are a class of terpenes that consist of two isoprene units and have the molecular formula C₁₀H₁₆. Monoterpenes may be linear (acyclic) or contain rings. Biochemical modifications such as oxidation or rearrangement produce the related monoterpenoids.

It has been described that cymene can be produced by alkylation of toluene with either propylene or isopropyl alcohol. A number of Friedel-Crafts catalysts, such as FeSO₄-HCl, AlCl₃, BF₃ or H₂SO₄ have been used for toluene isopropylation and solid acid catalysts have been used to produce p-cymene via alkylation of toluene with isopropyl alcohol (Ito et al., Hydrocarb. Process. 1973, 52(8), 89; Welstead et al., Encyclopedia Chem. Technol. 1978, 9, 544; Derfer et al., Encyclopedia Chem. Technol. 1978, 22, 709; Barman et al., Chemical Engineering Journal 2005, 114(1-3), 39-45). Methods for direct conversion of terpenes into cymene have also been described. These methods include for example conversion by acidic clays, oxidation with Cr(VI) compounds and transition metal based reactions. Vapour reactions using pure terpenes and Pd catalysts (Roberge et al., Appl. Catal. A, 2001, 215(1-2), 111-124) or Zn/Cr catalysts (Al-Waadani et al., Appl. Catal. A, 2009, 363(1-2), 153-156) have been reported.

Many of these methods are sensitive to sulphur and derivatives thereof, which deactivate the catalysts. Such methods are therefore not applicable to CST or other sulphur rich turpentine starting materials. Furthermore, many methods usually take place at high temperatures, such as above 300° C., at which temperatures CST tends to coke or polymerize. Both sensitivity to sulphur contamination and high operating temperatures make the prior art methods unsuitable for operation at sulphate- or sulphite-pulp mills.

WO2011/151526 describes a method for producing p-cymene from a starting material comprising at least one pinene. The reaction is catalyzed by a zeolite catalyst that is not sensitive to contamination by sulphur or derivatives thereof, so that crude sulphur turpentine (CST) obtained from wood pulping can be used as the starting material. However, the reaction takes place in the gas phase, at a temperature of preferably 300 to 350° C.

Significant amounts of turpentine are produced at pulp mills. For example, in the sulphate pulping of hard wood, about 0.5 kg turpentine/adt (air-dry tonne pulp) is formed. It would therefore be desirable to convert this by-product into potentially more valuable products, such as p-cymene. Since the prior art methods for producing p-cymene from terpenes are sensitive to sulphur contamination and generally demand high reaction temperatures, there is a continued need for an improved method for production of p-cymene from sulphur rich turpentine, in particular from crude sulphur turpentine (CST) produced at pulp mills.

### DETAILED DESCRIPTION OF THE INVENTION

It has surprisingly been discovered that cyclic monoterpenes can be oxidized to p-cymene in the presence of a Fe(III) salt as a catalyst. Importantly, this method is not sensitive to the presence of high levels of sulphur or sulphur derivatives in the starting material. The method is thus particularly suitable when the starting material comprises relatively high levels of sulphur, such as more than 0.5% (w/w), such as more than 1% (w/w), such as more than 2.5% (w/w), such as more than 5% (w/w), such as more than 10% (w/w), and turpentine from a thermo mechanical pulping process (TMP turpentine) or crude sulphate turpentine (CST) can readily be used as the starting material.

Another advantage of the invention is that the reaction can be carried out at a much lower temperature than in the methods of the prior art. In most of the prior art literature, conversion of cyclic monoterpenes to p-cymene takes place at temperatures above 180° C., and gas phase reactions at temperatures above 300° C. are not uncommon. In the method according to the present invention, the reaction takes place in the liquid phase and is highly efficient at reaction temperatures below 100° C. Although the reaction works at temperatures as low as 50° C., a reaction temperature of about 80-100° C. is more efficient.

Yet another advantage of the invention is that the reagents necessary for the conversion of cyclic monoterpenes into p-cymene are cheap materials, such as FeCl₃ and air.

The conversion of cyclic monoterpenes to p-cymene takes place via the terpinenes as the intermediates (see scheme 1). The terpinenes are formed from the cyclic monoterpenes by a Wagner-Meerwein rearrangement, which is mediated by a Lewis acid. It is therefore likely that the Fe³⁺ ions catalyze the isomerization of the cyclic monoterpenes to the terpinenes as well as the subsequent oxidation of the terpinenes to the resulting p-cymene. On the other hand, if sulphur rich turpentine is used as the starting material, such as crude sulphur turpentine, Fe³⁺ ions are likely to be reduced to Fe²⁺ ions by the sulphur derivatives present in the starting material. In that case, the isomerization of the cyclic monoterpenes to the terpinenes is probably mediated by Fe²⁺ ions acting as a Lewis acid. In the presence of an appropriate oxidant, such as oxygen or air, the Fe²⁺ ions can be oxidized to Fe³⁺ ions which can participate as catalysts in the subsequent oxidation of the terpinenes to the resulting p-cymene. Although the main product of the oxidation reaction is p-cymene, small amounts of m-cymene and trace amounts of o-cymene are also formed.

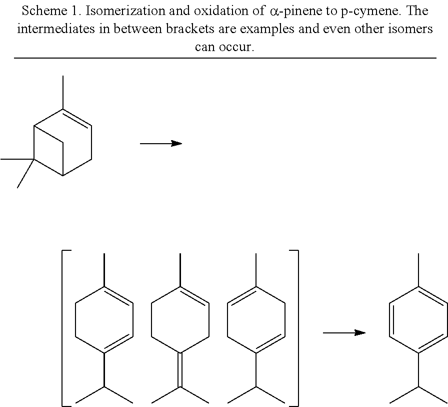

In a first aspect, the present invention relates to a method for production of p-cymene from a starting material comprising cyclic monoterpenes and/or terpinenes, wherein the starting material is converted to p-cymene in a liquid phase reaction in the presence of an Fe(III)-salt as a catalyst, in the presence of water and at pH 4 or below.

In one embodiment, the isomerization and oxidation reactions take place in a single process, catalyzed by the Fe(III) salt as outlined herein. As the starting material, a mixture comprising cyclic monoterpenes may be used, such as a mixture of cyclic monoterpenes. Preferably, the starting material is a mixture of α-pinene, β-pinene, 3-carene, sabinene, α-thujene, β-thujene and/or limonene. More preferably the starting material is a mixture comprising predominantly α-pinene, β-pinene, and/or 3-carene, and even more preferably the starting material is a mixture consisting essentially of α-pinene, β-pinene and/or 3-carene. Most preferably the starting material is crude sulphate turpentine (CST).

In another embodiment, the isomerization and oxidation reactions are performed in two different, subsequent processes. It has been observed that the oxidation reaction is faster and can produce p-cymene in higher yields if the starting material comprising cyclic monoterpenes is isomerized to the related terpinenes prior to the Fe(III)-catalyzed oxidation reaction. In that case, the starting material for the isomerization reaction should be a mixture comprising cyclic monoterpenes, as above, whereas the starting material for the subsequent oxidation reaction should comprise a mixture of terpinenes. Preferably, the starting material for the oxidation reaction is the mixture of terpinenes as obtained in the isomerization reaction.

Isomerization of the starting material comprising cyclic monoterpenes may be performed by heating the material in the presence of an appropriate Lewis acid such as FeCl₃, AlCl₃ or CrCl₃, or in the presence of an appropriate mineral acid such as aqueous sulphuric acid, aqueous phosphoric acid or aqueous hydrochloric acid. Even inhomogeneous materials such as acidic clays, zeolites and molybdenum heteropoly acids have been utilized for the isomerisation of terpenes. Preferably, the isomerization reaction is performed in the presence of diluted aqueous sulphuric acid (such as 5-50% in water, preferably 30-40% in water), and at a temperature between about 40 and about 180° C., preferably at a temperature between about 90 and about 120° C. The isomerized material may thereafter optionally be purified (e.g. washed with water) and/or isolated (e.g. distilled), and optionally also be stored. The isomerized material is then oxidized to p-cymene in a separate reaction, catalyzed by the Fe(III) salt as outlined herein.

The oxidation reaction, or the combined isomerization and oxidation reaction, is preferably performed at a temperature higher than about 50° C., such as higher than about 60° C., such as higher than about 70° C. In a preferred embodiment, the reaction is performed at a temperature between about 50 and about 130° C., preferably between about 70 and about 110° C., more preferably between about 75 and about 105° C., and even more preferably between about 80 and about 100° C. In a most preferred embodiment, the reaction is performed at about 90° C.

The oxidation reaction should be performed in the presence of at least a small amount of water. Although some water may already be present in the starting material (e.g. in turpentine or CST as remaining water from the pulping process) or in the catalyst (such as in FeCl₃*6H₂O), it is preferred that additional water is added to the reaction mixture. It is to be understood that water also may be added to the reaction mixture in the form of an aqueous solution of an acid, such as aqueous hydrochloric acid, or in the form of an aqueous solution of the catalyst.

A low pH is generally beneficial for the oxidation of the cyclic monoterpenes and/or terpinenes into p-cymene, since Fe(III) has the highest redox potential at low pH values. The oxidation reaction should therefore be performed at pH 4 or below. In a preferred embodiment, the reaction is performed at pH 3 or below, more preferably at pH 2 or below, and more preferably at pH 1.5 or below. In a most preferred embodiment the reaction is performed in the range of pH 0.5 to 3.0.

Aqueous solutions of Fe(III) salts generally have a pH value below 4. If necessary, the pH of the reaction mixture may be adjusted by the addition of an acid, such as aqueous hydrochloric acid. The pH of the reaction mixture is preferably adjusted to below 3.0, more preferably to below 2.0, most preferably to below 1.5. In one embodiment, the pH of the reaction mixture is in the range of 0.5 to 3.0.

In principle, the Fe(III) catalyst that is used in the reaction may be any Fe(III) salt that has sufficient solubility in the organic starting material and is able to form a stable Fe³⁺ complex that is active in the isomerization and oxidation of cyclic monoterpenes to p-cymene. The stability and activity of the Fe³⁺ complex may be influenced by the choice of ligands that coordinate to the Fe³⁺ ion. Such ligands can include inorganic ligands, such as, but not limited to, Cl⁻, SO₄²⁻ and SO₃²⁻, and organic ligands, such as, but not limited to, aliphatic carboxylic acids such as acetic acid, glycolic acid, propionic acid and lactic acid, and alkyl- or alkenyl succinic acid such as octadecenoic succinic acid, as well as combinations thereof. Alternatively, an Fe(II) salt may be used as the catalyst in the reaction, if the Fe(II) salt can be oxidized to an Fe(III) salt under the applied reaction conditions and form the soluble, stable and active Fe³⁺ complex in situ.

Preferably, the Fe(III) catalyst is FeCl₃ or FeCl₃*6H₂O. When dissolved in water, these salts dissociate as indicated below:

FeCl₃+3H₂O→Fe(OH)₃+3H⁺+3Cl⁻

Under these conditions, low levels of [FeCl₆]³⁻ are spontaneously formed. It is believed that this species is the active catalyst. It has a relatively high solubility in the organic phase, which is advantageous for the isomerization and oxidation reaction. In the presence of hydrochloric acid, the equilibrium is moved towards higher levels of [FeCl₆]³⁻.

During the oxidation of the organic material to p-cymene, the Fe³⁺ catalyst is reduced to Fe²⁺. If FeCl₃ is used as the catalyst, the reduced catalyst is probably FeCl₂ which has poor solubility in the organic phase. It will therefore transfer to the aqueous phase, where it needs to be reoxidized to the Fe³⁺ catalyst. The active [FeCl₆]³⁻ species can thereafter transfer back to the organic phase for oxidation of the organic material to p-cymene.

The Fe(III) salt should be added to the reaction mixture in a catalytic amount, such as at least 1% (w/w), such as at least 5% (w/w), preferably at least 10% (w/w), preferably at least 20% (w/w) of the total mass of the starting material. In a preferred embodiment, the amount of Fe(III) catalyst corresponds to between about 1 and about 70% (w/w), more preferably between about 5 and about 50% (w/w), even more preferably between about 20 and about 40% (w/w) of the total mass of the starting material.

In a preferred embodiment, the catalyst is FeCl₃ or FeCl₃*6H₂O and is added in an amount of between about 1 and about 70% (w/w), more preferably between about 5 and about 50% (w/w), even more preferably between about 20 and about 40% (w/w) of the total mass of the starting material. The use of relatively high amounts FeCl₃ is not a problem from an industrial point of view, since FeCl₃ is relatively cheap and furthermore can be re-oxidized and reused.

The catalyst may be added to the reaction mixture as a solid, (partially) dissolved or suspended in water, or (partially) dissolved or suspended in a solution of the acid in water. As an example, the catalyst may be added to the reaction mixture as partially dissolved in an aqueous solution of hydrochloric acid.

During the oxidation of the isomerized cyclic monoterpenes to p-cymene, the Fe³⁺ catalyst is reduced to Fe²⁺. If sulphur rich turpentine is used as the starting material, such as crude sulphur turpentine, the Fe³⁺ catalyst is also reduced to Fe²⁺ by the sulphur derivatives present in the starting material. Regeneration to Fe³⁺ may be achieved by re-oxidation of the formed Fe²⁺ with a suitable oxidant, such as oxygen or air. In one embodiment, the oxidant is oxygen. In another embodiment, the oxidant is air. In a preferred embodiment, the oxidant is air.

The starting material (i.e., the cyclic monoterpenes and/or terpinenes) and the formed p-cymene do not mix well with water. The method for production of p-cymene according to the invention is therefore typically a two-phase system, which consists of an aqueous lower phase and an organic upper phase containing the starting material (the cyclic monoterpenes) and/or the product (p-cymene). The Fe(III) catalyst preferably has relatively high solubility in the organic phase, but the reduced catalyst has poor solubility in the organic phase and transfers to the aqueous phase. This means that the reoxidation of the catalyst primarily must take place in the aqueous phase. Care should therefore be taken to bring the lower, aqueous phase in contact with oxygen or air. The skilled person is familiar with such techniques.

If the reaction is performed under under air or under an oxygen atmosphere, the aqueous phase may be brought into contact with the oxygen or air by vigorous stirring. Alternatively, the oxygen or air can be bubbled into the reaction mixture such that the oxygen or air is mixed with the aqueous phase. For safety reasons, it is sometimes preferred not to bring the oxygen or air into direct contact with the organic phase. The selective exposure of only the aqueous phase to oxygen or air reduces the risk for fire and explosions. In such case, part of the lower aqueous phase can repeatedly or continuously be withdrawn from the reaction mixture, brought into contact with oxygen or air, and subsequently reintroduced to the lower aqueous phase.

The redox potential of the Fe(III)/Fe(II) couple is pH dependent. Fe(III) has the highest redox potential at low pH values, whereas Fe(II) is most easily re-oxidized at higher pH values. The re-oxidation of the formed Fe(II) salts to the catalytic Fe(III) species is therefore slower under the acidic conditions applied to the oxidation reaction. If the pH value of the oxidation reaction is increased, the Fe(II) salts are more readily reoxidised to Fe (III), but the Fe(III) salts thus formed tend to precipitate as hydroxide species, such as Fe(OH)₃.

In case part of the aqueous phase is repeatedly or continuously withdrawn from the reaction mixture so that the catalyst can be re-oxidized by oxygen or air, the rate of re-oxidation of Fe(II) to Fe(III) can be accelerated by adjustment of the pH of the withdrawn aqueous phase. For example, a base such as NaOH may be added to increase the pH value to above 5. Once the catalyst has been re-oxidized to Fe(III), an appropriate acid such as hydrochloric acid may be added to the withdrawn aqueous phase in order to redissolve any precipitated catalyst, such as precipitated Fe(OH)₃, and to reform the active catalyst species [FeCl₆]³⁻.The aqueous phase containing the re-oxidized catalyst can thereafter be recirculated to the reaction mixture.

A frequently observed side-reaction in the conversion of cyclic monoterpenes to p-cymene is the formation of dimers, trimers and other polymer products. Both Lewis acids (such as AlCl₃) and mineral acids have been reported to polymerize cyclic monoterpenes into such oligomers and polymers. Although polymerization products are formed to some extent when using Fe(III) salts as a catalyst, the presence of the Fe(III) catalyst surprisingly does not lead to the formation of very large amounts of polymerization products. Small amounts of such polymerized products can be separated from the desired p-cymene by distillation. However, in order to further increase the yield of produced p-cymene, the polymerization reaction can be almost completely reduced if the reaction mixture (i.e., the mixture of cyclic monoterpenes and/or terpinenes) is diluted with a solvent that is miscible with the starting material and that is not reactive with the catalyst, such as aliphatic and/or aromatic hydrocarbons. This works particularly well if the reaction mixture is diluted with p-cymene. The use of p-cymene as a solvent is particularly beneficial since the added p-cymene does not need to be removed in a subsequent isolation step. Therefore, in one embodiment the reaction mixture is diluted with a solvent that is miscible with the starting material and that is not reactive with the catalyst. In a more preferred embodiment, the reaction mixture is diluted with an aliphatic and/or aromatic hydrocarbon solvent. In a most preferred embodiment, the reaction mixture is diluted with p-cymene.

In a second aspect, the invention relates to the method for production of p-cymene as outlined herein, wherein the method comprises the steps of:
i) providing a mixture of cyclic monoterpenes and/or terpinenes, optionally in the presence of a solvent;
ii) treating the mixture of step i) with an Fe(III)-catalyst and aqueous hydrochloric acid; and
iii) isolating the formed p-cymene from the reaction mixture.

In a preferred embodiment, the Fe(III)-catalyst is FeCl₃ or FeCI₃*6H₂O. In another preferred embodiment, the mixture of step i) is treated with an aqueous solution of the Fe(III)-catalyst. In yet another preferred embodiment, the isomerization and oxidation reactions are performed in two different processes and the starting material in step i) is a mixture of terpinenes as obtained in the isomerization reaction.

The formed p-cymene can be isolated from the crude reaction mixture using routine work-up procedures well-known to the skilled man, including steps such as, but not limited to, separation of the crude reaction mixture into an organic and an aqueous phase, washing of the organic phase with water and/or aqueous solutions, and drying of the organic phase. The p-cymene is then typically isolated from the organic reaction mixture by distillation. The organic reaction mixture will, in addition to the formed p-cymene, typically contain oligomer and polymer by-products as well as unreacted monoterpenes and terpinenes. Since the boiling point of p-cymene (177° C.) is close to the boiling point of most monoterpenes (the boiling points of α-pinene, β-pinene and 3-carene are about 157° C., 167° C. and 169° C., respectively), it can be difficult to isolate p-cymene from the reaction mixture by conventional distillation processes. The p-cymene can therefore conveniently be isolated from the reaction mixture by the method disclosed in WO 2013/120930. According to this method, sulphuric acid is added to the crude reaction mixture such that the concentration of sulphuric acid in the mixture is at least 0.5% (w/w), such as at least 3% (w/w), such as at least 5% (w/w). The addition of sulphuric acid leads to polymerization of the remaining monoterpenes into oligomers (e.g. diterpenes and triterpenes) which have a boiling point that is considerably higher than the boiling point of cymene, such as 50° C. higher or even 100° C. higher.

Following this treatment with sulphuric acid, the p-cymene can be distilled from the organic reaction mixture with higher purity.

In a third aspect, the invention relates to p-cymene obtained by the method according to the invention disclosed herein.

In a fourth aspect, the invention relates to the use of an Fe(III)-salt as a catalyst in a method for converting cyclic monoterpenes and/or terpinenes to p-cymene, wherein the conversion is achieved in the presence of water and at pH 4 or below. Preferably, the Fe(III)-salt used as the catalyst is FeCl₃ or FeCl₃*6H₂O.

The invention will now be described by the following examples which do not limit the invention in any respect. All cited documents and references are incorporated by reference.

### EXPERIMENTAL METHODS

Crude sulphur turpentine from Iggesund Mill, Holmen, was used as starting material in all experiments. The main components of the material are a-pinene (42%), β-pinene (12%) and 3-carene (46%), as determined by gas chromatography. The starting concentration of p-cymene in this material was 1.3 to 1.5%.

### EXAMPLES

### Example 1

### Isomerisation of Crude Sulphur Turpentine

A mixture of crude sulphur turpentine (500 mL) and diluted aqueous sulphuric acid (100 mL, 36%) was stirred at 110° C. for 5 hours. The aqueous layer was removed in a separatory funnel. The isomerized organic material was purified from heavier (polymerized) material by distillation under reduced pressure (5 hPa; 70-80° C.) and obtained in 76% yield.

### Example 2

### Oxidation of Mixture of Terpinenes to p-cymene

A solution of FeCl₃*6H₂O (31 g, 0.24 eq.) in water (60 mL) was added to a solution of the isomerized material of Example 1 (80 mL, 64 g) in p-cymene (79 mL, 63 g). The resulting mixture was heated at 90° C. and vigorously stirred under air for 1.5 hours. The product was isolated from the crude organic reaction mixture by distillation under reduced pressure (5 hPa; 70-80° C.) and obtained in 29% yield (corrected for the amount of p-cymene added as solvent).

### Example 3

### Oxidation of Mixture of Terpinenes to p-cymene

FeCl₃*6 H₂O (2.5 g) and diluted aqueous hydrochloric acid (10 mL, 13%) were added to the isomerized material of Example 1 (10 mL, 8 g). The resulting two-phase system was heated at 90° C. and vigorously stirred under air for 40 hours. The yield of p-cymene in the crude reaction mixture was 16.2%, as determined by gas chromatography.

1. Method for production of p-cymene, the method comprising:
converting a starting material comprising cyclic monoterpenes and/or terpinenes into p-cymene, wherein the starting material is converted to p-cymene in a liquid phase reaction using an Fe(III)-salt as a catalyst, in the presence of water and at pH 4 or below.

2. Method according to claim 1, wherein the starting material comprises cyclic monoterpenes.

3. Method according to claim 1, wherein the catalyst is FeCl₃ or FeCl₃*6H₂O.

4. Method according to claim 1, wherein the amount of catalyst is between about 20 and about 40% (w/w) of the total mass of the starting material.

5. Method according to claim 1, wherein the reaction is performed in the presence of hydrochloric acid.

6. Method according to claim 1, wherein the reaction is performed at a temperature between about 80 and about 100 ° C.

7. Method according to claim 1, wherein the sulphur content of the starting material is more than 2.5% (w/w).

8. Method according to claim 1, wherein the starting material is crude sulphate turpentine or TMP turpentine.

9. Method according to claim 1, wherein the starting material comprises terpinenes.

10. Method according to claim 9, wherein the isomerization of the cyclic monoterpenes to terpinenes is performed in a separate process.

11. Method according to claim 9, wherein the cyclic monoterpenes are isomerized to terpinenes by heating in the presence of aqueous sulphuric acid.

12. Method according to claim 1, wherein the reaction mixture is diluted with p-cymene.

13. Method according to claim 1, wherein the converting comprises the steps of:
i) providing a mixture of cyclic monoterpenes and/or terpinenes;
ii) treating the mixture of step i) with an Fe(III)-catalyst and aqueous hydrochloric acid; and
iii) isolating the formed p-cymene from the reaction mixture.

14. Method according to claim 13, wherein the catalyst is FeCl₃ or FeCl₃*6H₂O.

15. Method according to claim 13, wherein the mixture of step i) is treated with an aqueous solution of the Fe(III)-catalyst.

16. Method according to claim 13, wherein the isomerization and oxidation reactions are performed in two different processes and the starting material in step i) is a mixture of terpinenes as obtained in the isomerization reaction.

17. p-Cymene obtained by the method according to claim 1.

18. Use of an Fe(III)-salt as a catalyst in a method for converting cyclic monoterpenes and/or terpinenes to p-cymene, wherein the conversion is achieved in the presence of water and at pH 4 or below.

19. Method of claim 13, wherein providing the mixture of cyclic monoterpenes and/or terpinenes occurs in the presence of a solvent.
